(19) United States
(12) Patent Application Publication
(10) Pub. No.: US 2007/0243183 A1
(43) Pub. Date: Oct. 18, 2007

(54) Use of factor VIIa or factor VIIa equivalents for preventing or attenuating haemorrhage growth, and/or oedema generation following intracerebral haemorrhage

(75) Inventors: Nikolai Constantin Brun, Copenhagen N. (DK); Elisabeth Erhardtsen, Herlev (DK); Brett E. Skolnick, Philadelphia, PA (US)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/728,940

(22) Filed: Mar. 27, 2007

Related U.S. Application Data
(63) Continuation of application No. 11/169,933, filed on Jun. 29, 2005, now abandoned.

(30) Foreign Application Priority Data
Jun. 21, 2004 (DK) PA 2004 00972
Jun. 23, 2004 (DK) PA 2004 00981
Jun. 21, 2005 (EP) PCT/EP05/52890

(51) Int. Cl. A61K 38/48; A61K 38/45; A61P 43/00

(52) U.S. Cl. 424/94.64; 424/94.5

(57) ABSTRACT

The invention relates to the use of Factor VIIa or a Factor VIIa equivalent for the manufacture of a medicament for preventing complications in ICH patients.

### CROSS-REFERENCE TO RELATED APPLICATIONS

This patent application is a continuation of copending U.S. patent application Ser. No. 11/169,933, filed Jun. 29, 2005 [published as US Publication No. US-2006-0019894-A1] which is a continuation of International Application PCT/EP05/052890, filed Jun. 21, 2005; and claims the benefit of Danish Patent Application Nos. PA 2004 00972, filed Jun. 21, 2004, and PA 2004 00981, filed Jun. 23, 2004.

### FIELD OF THE INVENTION

The invention relates to the prevention of, or minimizing severity of complications in ICH patients.

### BACKGROUND OF THE INVENTION

Haemostasis is a complex physiological process which ultimately results in the arrest of bleeding. This is dependent on the proper function of three main components: blood vessels (especially the endothelial lining), coagulation factors, and platelets. Once a haemostatic plug is formed, the timely activation of the fibrinolytic system is equally important to prevent further unnecessary haemostatic activation. Any malfunction of this system (due to a reduced number, or molecular dysfunction, of the haemostatic components or increased activation of the fibrinolytic components) may lead to clinical bleeding such as, e.g., haemorrhagic diathesis of varying severity.

In most physiological situations, haemostasis is triggered by the interaction of circulating activated coagulation factor VII (FVIIa) with tissue factor (TF) subsequent to exposure of TF at the site of an injury. Endogenous FVIIa becomes proteolytically active only after forming a complex with TF. Normally, TF is expressed in the deep layers of the vessel wall and is exposed following injury. This ensures a highly localized activation of coagulation and prevents disseminated coagulation. TF also seems to exist in a non-active form, so-called encrypted TF. The regulation of encrypted versus active TF is still unknown.

Intracerebral hemorrhage (ICH) is a neurologic condition that occurs spontaneous and results in blood collecting in the intraparenchymal brain tissue. The results of an ICH have been demonstrated to result in significant morbidity and mortality. In recent years ICH has been shown to increase in volume in the hours following the initial insult. This occurs in at approximately 38% of patients suffering from ICH. The reason for the increase is unclear, but it is thought to be either through a continuous oozing of the original haematoma or through a complex process of rebleeds.

Days after the initial insult a zone of oedema can be identified on CT scans—surrounding the blood in the haematoma. The mechanism for oedema generation is also poorly understood but may be due to a combination of an inflammatory reaction in the tissue surrounding the clot as well as a direct mass effect of the clot exerting pressure on surrounding brain tissue. The impact of the isolated oedema can be significant but not evaluated in the context of significant haemorrhage but can have effects on the volume of compromised brain tissue after an ICH which has been estimated to be up to 3 times the actual volume of the haematoma. The importance of overall effected tissue volume would appear to be one of the strongest predictors of outcome after ICH. Thus there is clinical interest in reducing any haemorrhage expansion and in reducing and/or minimizing the total lesion volume (blood and resulting oedema).

Thus, there is a need in the art for improved methods and compositions for acute treatment of ICH, as well as for prevention and attenuation of later complications that result from ICH and from conventional modalities that are used to treat patients with ICH.

### BRIEF DESCRIPTION OF THE DRAWINGS

FIG. 1 is a graphic illustration of the percentage change in CT Volume over a 24 hour period.

### SUMMARY OF THE INVENTION

The invention provides the use of Factor VIIa or a Factor VIIa equivalent for the manufacture of a medicament for preventing or attenuating haemorrhage growth, and/or oedema generation following ICH. Typical patients for whom the medicament is used are those suffering from coagulopathic bleedings, including, without limitation, patients who have experienced spontaneous or traumatic ICH.

The invention also provides the use of Factor VIIa or a Factor VIIa equivalent for the manufacture of a medicament for increasing overall survival of a patient at day 90, preferably day 15 following the start of treatment. In another aspect, the invention provides the use of Factor VIIa or a Factor VIIa equivalent for the manufacture of a medicament for reducing the number of days of hospitalization of an ICH patient, including days in the Intensive Care Unit (ICU), bed confinement, and/or Quality of Life as measured by the European Quality of Life Scale (EuroQOL), or similar instruments, in the period from start of treatment (SOT) to day 90, preferably day 15 following the start of treatment or for reducing the risk of death in an ICH patient. In one embodiment, (i) an amount of 40, 80 or 160 μg/kg of Factor VIIa or Factor VIIa equivalent is administered to the patient at the start of treatment as a slow single bolus but in the setting of additional risk factors (e.g. anti-coagulant or anti-platelet treated patients may result in further dosing).

The invention also provides methods for preventing or attenuating one or more complications of ICH, which are carried out by administering to a patient an effective amount of Factor VIIa or a Factor VIIa equivalent. Typical patients have experienced spontaneous or traumatic ICH.

In some embodiments, the initial administering step is carried out within 4 hours of the occurrence of the ICH. In some embodiments, the method further comprises administering to the patient a second coagulation agent in an amount that augments the said Factor VIIa or Factor VIIa equivalent effect. Preferably, the second coagulation agent is a coagulation factor (including, without limitation, Factor VIII, Factor IX, Factor V, Factor XI, Factor XIII, and any combination thereof) or an antifibrinolytic agent (including, without limitation, PAI-1, aprotinin, ε-aminocaproic acid, tranexamic acid, or any combination thereof).

The invention also provides methods for reducing the number of days an ICH patient is hospitalized following ICH, which methods are carried out by administering to the patient an amount effective of Factor VIIa or a Factor VIIa equivalent to achieve the prevention or attenuation of hemorrhage growth, and/or oedema generation following ICH.

The invention also provides methods for reducing the risk of death in an ICH patient, which are carried out by administering an amount of Factor VIIa or a Factor VIIa equivalent to the patient for preventing or attenuating oedema generation following ICH.

The invention also provides methods for preventing or attenuating one or more complications of ICH in a majority of ICH patients, which are carried out by: (i) administering to a group of ICH patients an amount effective for achieving the prevention or attenuation of Factor VIIa or a Factor VIIa equivalent; and (ii) observing a reduction in the frequency of occurrence of one or more complications of ICH among the group of patients who received Factor VIIa or a Factor VIIa equivalent relative to the frequency of occurrence of said complications that would have been expected in the same group of patients who had not received said Factor VIIa or Factor VIIa equivalent.

### DETAILED DESCRIPTION OF THE INVENTION

The present invention provides methods and compositions that can be used advantageously to prevent or attenuate haemorrhage growth, and/or oedema generation following ICH, which patient may experience subsequent to their injury and/or as a result of medical interventions that may be used to treat their injuries. The methods are carried out by administering to an ICH patient, Factor VIIa or a Factor VIIa equivalent, in a manner that is effective for preventing or attenuating haemorrhage growth, oedema formation as well as one or more complications related to ICH. A manner effective for preventing or attenuating haemorrhage growth, oedema formation and the subsequent complications may comprise administering a predetermined amount of Factor VIIa or a Factor VIIa equivalent, and/or utilizing a particular dosage regimen, formulation, mode of administration, combination with other treatments, and the like. The efficacy of the methods of the invention in reducing haemorrhage growth, oedema formation or in preventing complications of ICH may be assessed using one or more conventional imaging methods (e.g., CT, MRI scanning) or by use of parameters that evaluate complications (see below). Complications that may be prevented by the methods of the invention, or whose severity may be attenuated, include, without limitation, haemorrhage growth, oedema generation, and decreased quality of life including death caused by one or more of these syndromes.

Patient Selection:

Patients who may benefit by use of the methods of the present invention include, without limitation, patients who have suffered from spontaneous or traumatic ICH. Spontaneous ICH includes patients suffering an intracerebral bleed usually associated with the occurrence of advanced age, hypertension, or deposition of amyloid in the cerebral vasculature. ICH usually results from the rupture of a single vessel causing extensive damage to the surrounding brain tissue adjacent to the damaged vessel. Traumatic ICH may be associated with accidents resulting from e.g. motor vehicle accidents or fall from a height. The resulting contusion to the head may lead to the rupture of one or more intracerebral or extracerebral (but intracranial) vessels. Many intracranial (but extracerebral) bleedings are evacuated surgically already in the acute phase, whereas the intracerebral lesions more often are inaccessible to direct evacuation as the evacuation itself would cause significant damage to the brain tissue.

Bleeding refers to extravasation of blood from any component of the circulatory system and encompasses any bleeding (including, without limitation, excessive, uncontrolled bleeding, i.e., hemorrhaging) in connection with ICH. In one series of embodiments, the excessive bleeding is caused by spontaneous ICH; in another it is caused by traumatic ICH.

The methods of the present invention can be applied advantageously to any patient who has suffered spontaneous or traumatic ICH that, if left untreated, would result in a significant growth of the haemorrhage and in associated oedema and/or complications.

In one series of embodiments, patients treated according to the invention do not suffer from a bleeding disorder, whether congenital or acquired, such as, e.g., Haemophilia A, B. or C.

In different embodiments of the invention, patients may be excluded from treatment if they have been diagnosed with a congenital bleeding disorder.

In different embodiments of the invention, patients may be excluded from treatment if they have been previously treated with anticoagulation therapies.

Factor VIIa and Factor VIIa Equivalents:

In practicing the present invention, any Factor VIIa or equivalent may be used that is effective in preventing complications when administered to an ICH patient. In some embodiments, the Factor VIIa is human Factor VIIa, as disclosed, e.g., in U.S. Pat. No. 4,784,950 (wild-type Factor VII). The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

Factor VIIa equivalents include, without limitation, Factor VII polypeptides that have either been chemically modified relative to human Factor VIIa and/or contain one or more amino acid sequence alterations relative to human Factor VIIa. Such equivalents may exhibit different properties relative to human Factor VIIa, including stability, phospholipid binding, altered specific activity, and the like.

In one series of embodiments, a Factor VIIa equivalent includes polypeptides that exhibit at least about 10%, preferably at least about 30%, more preferably at least about 50%, and most preferably at least about 70%, of the specific biological activity of human Factor VIIa. For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa or a Factor VIIa equivalent to produce of Factor Xa in a system comprising TF embedded in a lipid membrane and Factor X. (Persson et al., J. Biol. Chem. 272:19919-19924, 1997); (ii) measuring Factor X hydrolysis in an aqueous system (see, Example 5 below); (iii) measuring the physical binding of Factor VIIa or a Factor VIIa equivalent to TF using an instrument based on surface plasmon resonance (Persson, FEBS Letts. 413:359-363, 1997) and (iv) measuring hydrolysis of a synthetic substrate by Factor VIIa and/or a Factor VIIa equivalent.

Examples of factor VII equivalents include, without limitation, wild-type Factor VII, L305V-FVII, L305V/M306D/D309S-FVII, L305I-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, and K316Q/L305V/V158T/E296V/M298Q/K337A-FVII.

Preparations and Formulations:

The present invention encompasses therapeutic administration of Factor VIIa or Factor VIIa equivalents, which is achieved using formulations that comprise Factor VIIa preparations. As used herein, a “Factor VII preparation” refers to a plurality of Factor VIIa polypeptides or Factor VIIa equivalent polypeptides, including variants and chemically modified forms, that have been separated from the cell in which they were synthesized, whether a cell of origin or a recombinant cell that has been programmed to synthesize Factor VIIa or a Factor VIIa equivalent.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989. Following purification, the preparation preferably contains less than about 10% by weight, more preferably less than about 5% and most preferably less than about 1%, of non-Factor VII proteins derived from the host cell.

Factor VII and Factor VII-related polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like. The resulting activated Factor VII may then be formulated and administered as described below.

Pharmaceutical compositions or formulations for use in the present invention comprise a Factor VIIa preparation in combination with, preferably dissolved in, a pharmaceutically acceptable carrier, preferably an aqueous carrier or diluent. A variety of aqueous carriers may be used, such as water, buffered water, 0.4% saline, 0.3% glycine and the like. The preparations of the invention can also be formulated into liposome preparations for delivery or targeting to the sites of injury. Liposome preparations are generally described in, e.g., U.S. Pat. Nos. 4,837,028, 4,501,728, and 4,975,282. The compositions may be sterilised by conventional, well-known sterilisation techniques. The resulting aqueous solutions may be packaged for use or filtered under aseptic conditions and lyophilised, the lyophilised preparation being combined with a sterile aqueous solution prior to administration.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

Treatment Regimen:

In practicing the present invention, Factor VIIa or the Factor VIIa equivalent may be administered to a patient as a single dose comprising a single-dose-effective amount for preventing haemorrhage growth, and/or oedema formation and/or for treating complications, or in a staged series of doses which together comprise an effective amount for preventing or treating complications. An effective amount of Factor VIIa or the Factor VIIa equivalent (see below) refers to the amount of Factor VIIa or equivalent which, when administered in a single dose or in the aggregate of multiple doses, or as part of any other type of defined treatment regimen, produces a measurable statistical improvement in outcome, as evidenced by at least one clinical parameter associated with ICH and/or its complications (see below). When Factor VIIa equivalents are administered, an effective amount may be determined by comparing the coagulant activity of the Factor VIIa equivalent with that of Factor VIIa and adjusting the amount to be administered proportionately to the predetermined effective dose of Factor VIIa.

Administration of Factor VIIa or a Factor VIIa equivalent according to the present invention is preferably initiated within about 4 hours after occurrence of the ICH such as, e.g., within about 3 hours, within about 2 hours, or within about 1 hour.

Administration of a single dose refers to administration of an entire dose of Factor VIIa or the Factor VIIa equivalent as a slow bolus over a period of less than about 5 minutes. In some embodiments, the administration occurs over a period of less than about 2.5 minutes, and, in some, over less than about 1 min. Typically, a single-dose effective amount comprises at least about 40 μg/kg human Factor VIIa or a corresponding amount of a Factor VIIa equivalent, such as, at least about 50 μg/kg, 75 μg/kg, or 90 μg/kg, or at least 160 μg/kg Factor VIIa.

It will be understood that the effective amount of Factor VIIa or Factor VIIa equivalent, as well as the overall dosage regimen, may vary according to the patient's haemostatic status, which, in turn, may be reflected in one or more clinical parameters, including, e.g., relative levels of circulating coagulation factors; amount of blood lost; rate of bleeding; haematocrit, and the like. It will be further understood that the effective amount may be determined by those of ordinary skill in the art by routine experimentation, by constructing a matrix of values and testing different points in the matrix.

For example, in one series of embodiments, the invention encompasses (i) administering a first dose of Factor VIIa or a Factor VIIa equivalent; (ii) assessing the patient's coagulation status after a predetermined time; and (iii) based on the assessment, administering a further dose of Factor VIIa or Factor VIIa equivalent if necessary. Steps (ii) and (iii) may be repeated until satisfactory haemostasis is achieved.

According to the invention, Factor VIIa or a Factor VIIa equivalent may be administered by any effective route, including, without limitation, intravenous, intramuscular, subcutaneous, mucosal, and pulmonary routes of administration. Preferably, administration is by an intravenous route.

Combination Treatments:

The present invention encompasses combined administration of an additional agent in concert with Factor VIIa or a Factor VIIa equivalent. In some embodiments, the additional agent comprises a coagulant, including, without limitation, a coagulation factor such as, e.g., Factor VIII, Factor IX, Factor V, Factor XI, or Factor XIII; or an inhibitor of the fibrinolytic system, such as, e.g., PAI-1, aprotinin, ε-aminocaproic acid or tranexamic acid.

It will be understood that, in embodiments comprising administration of combinations of Factor VIIa with other agents, the dosage of Factor VIIa or Factor VIIa equivalent may on its own comprise an effective amount and additional agent(s) may further augment the therapeutic benefit to the patient. Alternatively, the combination of Factor VIIa or equivalent and the second agent may together comprise an effective amount for preventing complications associated with ICH. It will also be understood that effective amounts may be defined in the context of particular treatment regimens, including, e.g., timing and number of administrations, modes of administrations, formulations, etc.

Treatment Outcomes:

The present invention provides methods and compositions for preventing or attenuating haemorrhage growth, and/or oedema generation following ICH, as well as preventing and/or attenuating one or more complications of ICH. Complications include, without limitation, Cerebral Oedema, decreased quality of life including death caused by one or more of these syndromes.

In practicing the present invention, the severity of ICH and its complications may be assessed using conventional methods, such as, e.g., Imaging by CT or MR scans or the Clinical assessment scores (Scores) described herein. Assessments may be performed at least about 15 days from the start of treatment according to the invention, such as, e.g., at least about 30 days, at least about 40 days, or at least about 90 days from the start of treatment.

Organ damage or organ failure encompasses, without limitation, damage to the structure and/or damage to the functioning of the organ i.e. the brain, defined but not limited to cerebrum, cerebellar, pons, medulla, brain stem, spinal cord or surrounding tissues. Examples of organ damage include, but are not limited to, morphological/structural damage and/or damage to the functioning of the organ such as, for example accumulation of excess fluid or proteins. The terms “organ injury”, “organ damage” and “organ failure” may be used interchangeably. Normally, organ damage results in organ failure. By organ failure is meant a decrease in organ function compared to the mean, normal functioning of a corresponding organ in a normal, healthy person. The organ failure may be a minor decrease in function (e.g., 80-90% of normal) or it may be a major decrease in function (e.g., 10-20% of normal); the decrease may also be a complete failure of organ function. Organ failure includes, without limitation, decreased biological functioning, e.g., due to tissue necrosis, fibrin deposition, haemorrhage, oedema, or inflammation and/or the complications that occur in response to these failures like brain herniation. Organ damage includes, without limitation, tissue necrosis, fibrin deposition, haemorrhage, oedema, or inflammation.
Methods for testing organ function and efficiency, and suitable biochemical or clinical parameters for such testing, are well known to the skilled clinician.
Such markers or biochemical parameters of organ function are, for example:
Brain perfusion: Measurements of Cerebral blood flow
Brain metabolism: Measurements of cerebral oxygen extraction or direct measurements of cerebral metabolic rate of oxygen (e.g., by MRS, PET or SPECT scans).
Measurement of other substrates than oxygen such as glucose are also included.
Brain integrity: MRI (any and all standarized protocol sequences), CT, CTA, MRA
Brain cell electrical function as measured by EEG
Brain function by well established neurological tests (e.g., Microdialysis, Transcranial Doppler)

Methods for testing for coagulopathy and inflammation are also well known to the skilled clinician. Such markers of a coagulapathic state are, for example, PTT, Fibrinogen depletion, elevation in TAT complexes, ATIII activity, IL-6, IL-8, or TNFR-1.

In the present context, prevention includes, without limitation, the attenuation, elimination, minimization, alleviation or amelioration of one or more symptoms or conditions associated with ICH and/or its complications, including, but not limited to, the prevention of further damage to and/or failure of the effected organ already subject to some degree of organ failure and/or damage, as well as the prevention of damage and/or failure of further organs not yet subject to organ failure and/or damage. Examples of such symptoms or conditions include, but are not limited to, morphological/structural damage and/or damage to the functioning of organs such as, but not limited to, brain and surrounding organs. Examples of such symptoms or conditions include, but are not limited to, morphological/structural damage and/or damage to the functioning of the organ(s) such as, for example, accumulation of proteins or fluids due to mass effect of the haematoma or from resulting inflammatory reactions in the surrounding tissue, tissue necrosis, fibrin deposition, haemorrhage, oedema, or inflammation.

Attenuation of organ failure or damage encompasses any improvement in organ function as measured by at least one of the well known markers of function of said organ (see below) compared to the corresponding value(s) found in ICH patients not being treated in accordance with the present invention.

In various embodiments, haematoma growth is reduced by at least 5%, such as, e.g., 10%, 20%, 30%, 40%, 50%, 60%, or at least 70% compared to haematoma growth in patients not treated with Factor VIIa or a Factor VIIa equivalent in accordance with the present invention. In various embodiments, oedema generation is reduced by at least 5%, such as, e.g., 10%, 20%, 30%, 40%, 50%, 60%, or at least 70% compared to oedema generation in patients not treated with Factor VIIa or a Factor VIIa equivalent in accordance with the present invention. In other embodiments, total haemorrhage volume (ICH+IVH) is reduced by at least 5%, such as, e.g., 10%, 20%, 30%, 40%, 50%, 60%, or at least 70% compared to total haemorrhage volume in patients not treated with Factor VIIa or a Factor VIIa equivalent in accordance with the present invention.

Measurement of ICH Severity and/or Complications:

Following are non-limiting examples of methods for assessing the incidence and severity of complications of ICH.

The Glasgow Coma Score (GCS; Teasdale and Jennett, The Lancet 13; 2(7872):81-84, 1974) is determined by use of the attached instrument or its equivalents (Appendix 1; below)

The modified Rankin Scale (mRS; Bonita and Beaglehole, Stroke 1988 December; 19(12): 1497-1500) is determined by use of the attached instrument or its equivalents (Appendix 2; below)

The Barthel Index (BI; Mahoney and Barthel, Maryland State Medical Journal 1965; 14:56-61) is determined by use of the attached instrument or its equivalents (Appendix 3; below).

The NIH Stroke Scale (NIHSS; Brott et al., 1989) is determined using the attached instrument or its equivalent (Appendix 4; below).

The Glasgow Outcome Scale extended version (GOSe, Lindsay et al., Journal of Neurotrauma; 15 (8): 573-580, 1998) is determined by use of the attached instrument or its equivalents (Appendix 5; below).

Information of scales and assessment tools, including the five above-mentioned well-known instruments, is found at The Internet Stroke Center Washington, University School of Medicine, Department of Neurology (www.strokecenter.org).

Other Indices of Treatment:

The efficacy of the methods of the present invention may also be assessed using other clinical parameters, including, without limitation, reduction in any one or more of the following parameters relative to a similar patient who has not been administered Factor VIIa or a Factor VIIa equivalent according to the invention: an improvement in neurological outcome as determined by the NIHSS, the mRS, the E-GOS, the GCS or the BI scales as described above.; a decrease in the number of days of hospitalization after suffering an ICH, including a decrease in the number of days that a patient may spend in an intensive care unit (ICU) and a decrease in the number of days in which certain interventions (such as, e.g., a ventilator) are required. Non-limiting examples of outcomes include: (i) an improvement in scores on the NIHSS by at least 1, 2, 4, 8, 10, or 20 points on the scale (ii) an improvement on the mRS scale by at least 1, 2, 3, or 4 points on the scale; (iii) an improvement on the BI scale by at least 5, 10, 15, 20 or 30 points on the scale; (iv) an improvement on the 8 point GOS by at least 1, 2, 3, 5, or 7 points on the scale (v) a decrease in ICU days by 1 day, 2 days, or 4 days; (vi) a reduction on the number of days on a ventilator by 1 day, 2 days, or 4 days; (vii) a reduction in the total days of hospitalization by 2 days, 4 days, or 8 days.

### Various Embodiments of the Invention

The present invention encompasses the use of Factor VIIa or a Factor VIIa equivalent for the manufacture of a medicament for preventing or attenuating haemorrhage growth, and/or oedema generation following ICH as well as preventing or attenuating one or more complications of ICH. Non-limiting examples of complications include: cerebral oedema and poor neurological outcome after ICH, and death. In some embodiments, the patient is suffering from spontaneous ICH and in some from traumatic ICH.

In some embodiments, the medicament comprises at least about 160 μg/kg of Factor VIIa or a corresponding amount of a Factor VIIa equivalent. In some embodiments, the medicament is for administration in a first dose containing at least about 160 μg/kg.

In another aspect, the invention encompasses the use of Factor VIIa or a Factor VIIa equivalent for the manufacture of a medicament for reducing the number of days an ICH patient is hospitalized following symptom onset or injury onset. In some embodiments, the medicament is for reducing the number of days an ICH patient spends in an Intensive Care Unit (ICU) following injury or symptom onset.

In another aspect, the invention encompasses the use of Factor VIIa or a Factor VIIa equivalent for the manufacture of a medicament for improving brain function in an ICH patient. In some embodiments, the medicament is for reducing the amount of brain oedema and the associated risks of further neurological deterioration associated with such oedema in an ICH patient. In some embodiments, the medicament is for reducing the risk of progression from brain injury.

In another aspect, the invention encompasses the use of Factor VIIa or a Factor VIIa equivalent for the manufacture of a medicament for reducing the risk of death in an ICH patient.

In some embodiments, the medicament further comprises a second coagulation agent in an amount that augments said preventing or attenuating by the Factor VIIa or Factor VIIa equivalent. In some embodiments, the second coagulation agent is selected from the group consisting of a coagulation factor and an antifibrinolytic agent. Non-limiting examples of a coagulation factor include Factor VIII, Factor IX, Factor V, Factor XI, Factor XIII, and any combination of the foregoing; and non-limiting examples of the antifibrinolytic agent include PAI-1, aprotinin, ε-aminocaproic acid, and tranexamic acid.

In another aspect, the invention provides kits of parts for preventing or attenuating haemorrhage growth, and/or oedema generation following ICH as well as preventing or attenuating one or more complications of ICH, comprising
(i) A medicament comprising Factor VIIa or a Factor VIIa equivalent; and
(ii) Instructions for Use describing that:
   a. A first dose containing at least about 50, preferably at least about 160 ug/kg Factor VIIa or a corresponding amount of a Factor VIIa equivalent, should be administered at the start of treatment;
   b. A second dose may be needed and may be in the amounts of 40, 80 or 160 μg/kg Factor VIIa or a corresponding amount of a Factor VIIa equivalent should be administered one hour after the start of treatment.

In another aspect, the invention provides methods for preventing or attenuating haemorrhage growth, and/or oedema generation following ICH as well as preventing or attenuating one or more complications of ICH, the methods comprising administering to a patient in need of said preventing or attenuating an effective amount for the preventing or attenuating of Factor VIIa or a Factor VIIa equivalent. Non-limiting examples of complications include: brain death, brain herniation, respiratory compromise secondary to brain herniation and any other related complications secondary to brain dysfunction. In some embodiments, the patient is suffering from spontaneous ICH and in others from traumatic ICH.

In some embodiments, the effective amount comprises at least about 40 μg/kg of Factor VIIa or a corresponding amount of a Factor VIIa equivalent. In some embodiments, a first amount of at least about 160 μg/kg Factor VIIa or a corresponding amount of a Factor VIIa equivalent is administered at the start of treatment, and a second amount of about 40, 80 or 160 μg/kg of Factor VIIa or a corresponding amount of a Factor VIIa equivalent is administered to the patient one hour after the start of treatment. In some embodiments, the method further comprises administering to the patient a second coagulation agent in an amount that augments the preventing or attenuating by Factor VIIa or a Factor VIIa equivalent. In some embodiments, the second coagulation agent is a coagulation factor or an antifibrinolytic agent. Non-limiting examples of a coagulation factor include Factor VIII, Factor IX, Factor V, Factor XI, Factor XIII, and any combination of the foregoing; and non-limiting examples of an antifibrinolytic agent include PAI-1, aprotinin, ε-aminocaproic acid, and tranexamic acid.

In another aspect, the invention provides methods for reducing the number of days an ICH patient is hospitalized following spontaneous ICH or traumatic ICH, which are carried out by administering to the patient an effective amount for the reduction of Factor VIIa or a Factor VIIa equivalent.

In another aspect, the invention provides methods for reducing the number of days an ICH patient spends in an Intensive Care Unit (ICU) following injury or symptom onset, which are carried out by administering to the patient an effective amount for the reduction of Factor VIIa or a Factor VIIa equivalent.

In another aspect, the invention provides methods for improving brain function in an ICH patient, which are carried out by administering to the patient an effective amount for the improving of Factor VIIa or a Factor VIIa equivalent.

In another aspect, the invention provides methods for reducing the risk of developing complications of brain dysfunction including, but not limited to brain herniation, brain infarction in an ICH patient, which methods are carried out by administering to the patient an effective amount for the reducing of Factor VIIa or a Factor VIIa equivalent. In some embodiments, the invention provides methods for reducing the risk of progression from brain injury to brain death.

In another aspect, the invention provides methods for reducing the risk of death in an ICH patient, which are carried out by administering to the patient an effective amount for the reducing of Factor VIIa or a Factor VIIa equivalent.

In another aspect, the invention provides methods for preventing or attenuating haemorrhage growth, and/or oedema generation following ICH, which are carried out by intentionally administering to a patient in need of the preventing or attenuating an effective amount for the preventing or attenuating of Factor VIIa or a Factor VIIa equivalent for the purpose of preventing or attenuating the haemorrhage growth, and/or oedema generation.

In another aspect, the invention provides methods for preventing or attenuating haemorrhage growth, and/or oedema generation following ICH in a majority of spontaneous ICH or traumatic ICH patients, which are carried out by (i) administering to a group of ICH patients an effective amount for the preventing or attenuating of Factor VIIa or a Factor VIIa equivalent; and (ii) observing a reduction in the frequency of occurrence of one or more complications of ICH among the group of patients relative to the frequency of occurrence of the complications that would have been expected in the same group of patients who had not received the Factor VIIa or Factor VIIa equivalent.

The following examples are intended as non-limiting illustrations of the present invention.

### EXAMPLES

General Methods

Methods of Measurement.

Haematoma, Intraventricular Haemorrhage (IVH) and oedema volumes (mm³) were measured by computed tomography scan (CT scan) using Analyze™ software (Biomedical Imaging Resource at the Mayo Foundation) equipped with a ROI (region of interest) module.

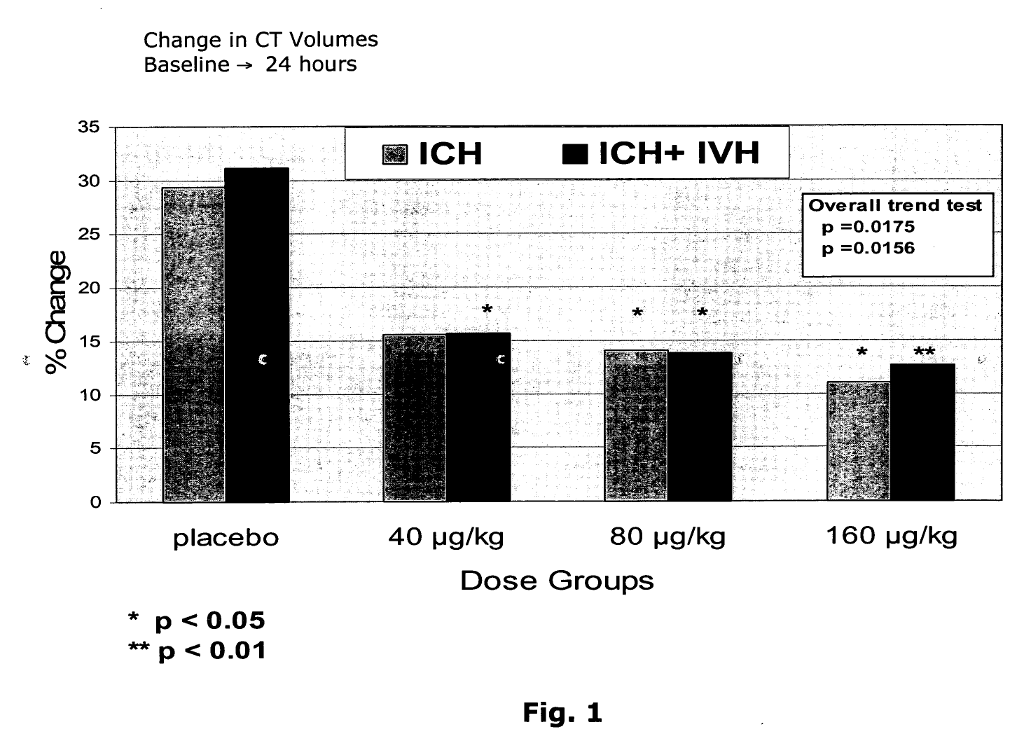

Haematoma, Intraventricular Haemorrhage (IVH) and oedema volumes (mm³) were calculated by a neuroradiologist blinded to treatment allocation at a centralized location by tracing the perimeter of appropriate high- or low-attenuation zone in each involved cross-sectional image (“slice”) using a computerized imaging system. All measurements will be made using the Region of Interest (ROI) module within the Analyze™ software. The reviewer will define each target area using the semi-automated segmentation and/or freehand tracing tools. Each defined area will be editable to aid the reviewer to include only the area of interest, which is represented as a ROI on the image (see FIGS. 1a and 1b). This procedure will be reproduced for each slice as well as each separate target area the reviewer determines is necessary.

Once all areas have been defined by the reviewer, Analyze™ software will be used to calculate statistics of each ROI. This is to include the area of the ROI, defined in mm² and the volume of the ROI, defined in mm³. The volume of the ROI is calculated by multiplying the slice thickness of the acquisition by the area. Since an object may include more then one slice, the ROI volume for each slice is displayed in a “Stat Log Region of Interest” window. This information in this window is saved as an ASCII file and directly imported into the Blind Read Database. Once all volumes have been imported into the Blind Read Database, the reviewer-defined regions are stored as object maps and saved. An object map is simply a copy of the volume with defined areas/structures.

The reviewer will use the Region of Interest (ROI) module from the Analyze™ software to measure the following:
The volume of ICH.
The volume of Intraventricular Haemorrhage (IVH).
The total volume of Perihematoma oedema.

Based upon the above measurements the following will be calculated:
The change in ICH volume from the screening to the 24 hour CT scan expressed in mm³ and percent.
   Calculated as change in percent=[(ICH volume at 24 hours−ICH volume at screening)/ICH volume at screening]100.
   Calculated as change in millimeters³=ICH volume at 24 hours−ICH volume at screening
Ratio of oedema/ICH volume at each CT scan
   Ratio=volume of oedema/ICH volume
Total Haemorrhage at each CT scan.
   Calculated as Total Haemorrhage=ICH+IVH

The following calculations will be used on CT scans submitted using multi-slice thickness techniques. (A smaller slice thickness used through the posterior fossa with a transition to a larger slice thickness through to the vertex.)
1=Smaller Slice Thickness Acquisition.
2=Larger slice Thickness Acquisition.
   Calculated as Slice Thickness (mm)/Slice Spacing (mm)=X/[Table Position 1 (mm)+½ Slice Spacing 1 (mm)]−[Table Position 2 (mm)−½ Slice Spacing 2 (mm)] Volume of Gap/Overlap in millimeters³=X(Area of final slice in 1 in millimeters²).

### Working Examples

### Example 1

### Efficacy of Factor VIIa Given to ICH Patients

Objectives

To evaluate the efficacy and safety of NovoSeven® in preventing early haematoma growth in acute Intracerebral Haemorrhage (ICH). Treatment is intended to be administered as soon as possible following injury or symptom onset but can be delayed due to transportation and/or mitigating medical circumstances. Timing should be in the order of hours not days following onset of injury or symptoms.

Trial Design:

The trial was a randomised, double-blind, multi-centre, multi-national, placebo-controlled efficacy and safety trial with four treatment arms: doses of 40, 80 and 160 μg/kg against placebo.

Trial Population:

Number of subjects to be studied: 400 (four hundred) patients with ICH.

Inclusion Criteria

1. Spontaneous ICH (incl brainstem and cerebellum) diagnosed by CT scanning within 3 hours of onset.
2. Male or female subjects, age ≧18 years.
3. Signed informed consent form, or in countries where waiver of informed consent is allowed by IRB/IEC, a completed waiver form.
   Exclusion Criteria
1. Time of onset of symptoms of ICH unknown or >3 hours.
2. Patients with secondary ICH related to infarction, tumour, haemorrhagic infarction, cerebrovenous thrombosis, aneurysm, arteriovenous malformations (AVM), thrombolysis or severe trauma.
3. Surgical haematoma evacuation planned or performed within 24 hours of onset.
4. Deep Coma (GCS 3-5) at the time of admission.
5. Known oral anti-coagulant use (unless INR documented below 1.4). Aspirin use is not an exclusion criterion.
6. Known thrombocytopenia (unless platelets documented above 50,000/μL).
7. Preexisting disability (i.e must have mRS score 0-2 before stroke).
8. Any history of haemophilia or other coagulopathy.
9. Acute myocardial ischaemia, acute septicaemia, acute crush injury, acute haemorrhagic disseminated intravascular coagulation, or acute thrombotic stroke.
10. Pregnancy.
11. History of angina, myocardial infarction, ischaemic stroke, previous limb amputation due to vascular disease, or claudication within last 30 days.
12. Known or suspected allergy to trial product or related products.
13. Previous participation in this trial.
14. Participation in ANY investigational drug or device trial within 30 days of entry into the trial.
   Assessments:
   Efficacy Endpoints:
   The primary efficacy endpoint is the change in ICH volume as measured by CT head scans from prior to dosing to 24 hours after the baseline scan.
   Secondary efficacy endpoints are differences between groups on the modified Rankin Scale (mRS), the Barthel Index (BI), the Glasgow Coma Scale (GCS), the 8-point Glasgow Outcome Scale (GOS), The EuroQOL scale, and the National Institute of Health's Stroke Scale (NIHSS) over the duration of the trial.
      Safety Endpoints:
   The occurrence of adverse events until discharge and serious adverse events until the End of Trial Form is completed.
   Safety laboratory coagulation parameters (Fibrinogen and Fragment1+2) from prior to dosing to one hour post-dosing.
   Exacerbation of brain oedema (oedema/ICH-volume ratio) as assessed using Head CT scan at 72 hours post-dosing.
      Trial Product(S):

Activated recombinant human factor VII (rFVIIa/NovoSeven®) and placebo are supplied by Novo Nordisk A/S, Denmark as freeze-dried powders to be reconstituted with water for injection.

### Example 2

### Factor VIIa Administration to ICH Patients

The ITT population included 399 patients, after 1 patient was removed after having withdrawn consent.

ICH haematoma growth (ITT): % change in baseline to 24 hours

| Placebo | 40 μg/kg | 80 μg/kg | 160 μg/kg | P value |
|---|---|---|---|---|
| 29% | 16% | 14% | 11% | 0.0175 |
| | p = 0.0710 | p = 0.0486 | P = 0.0150 | (overall |
| | | | | test for trend) |
| | | | | 0.0112 |
| | | | | (combined |
| | | | | NovoSeven ® |
| | | | | vs placebo) |

Change in CT volumes: % change in baseline to 24 hours (illustrated also in Figure 1)

| Placebo | 40 μg/kg | 80 μg/kg | 160 μg/kg | P value |
|---|---|---|---|---|
| ICH 29% | ICH 16% | ICH 14% | ICH 12% | Overall trend test: ICH |
| | | P < 0.05 | P < 0.05 | p = 0.0175 |
| ICH + IVH 31% | ICH + IVH 16% | ICH + IVH 14% | ICH + IVH 13% | ICH + IVH |
| | P < 0.05 | P < 0.05 | P < 0.01 | p = 0.0156 |

ICH: intracerebral haemorrhage
IVH: intraventricular haemorrhage
Total Haemorrhage = ICH + IVH

### Example 3

### Factor VIIa administration to ICH patients

Secondary Efficacy, Haemorrhage Endpoints Analysis of Edema Volume at 72 Hours after Baseline ITT Population

| | Mean | SE | 98.3% CI | p-value |
|---|---|---|---|---|
| Test for trend | | | | 0.0014 |
| 40 ug/kg - Placebo | −3.90 | 2.51 | [−9.95; 2.15] | 0.1219 |
| 80 ug/kg - Placebo | −6.05 | 2.62 | [−12.35; 0.25] | 0.0216 |
| 160 ug/kg - Placebo | −7.97 | 2.56 | [−14.13; −1.82] | 0.0020 |
| rFVIIa Combined - Placebo | −5.92 | 2.10 | [−10.98; −0.86] | 0.0051 |

Change in volumes are analysed by a generalised linear mixed model with subject and reader as random effects and volume at baseline, time from stroke to baseline CT, and time from baseline CT to dosing as fixed effects covariates

Secondary Efficacy, Haemorrhage Endpoints Analysis of Total Edema Volume (ICH+IVH+Edema) at 72 Hours after Baseline ITT Population

| | Mean | SE | 98.3% CI | p-value |
|---|---|---|---|---|
| Test for trend | | | | 0.0006 |
| 40 ug/kg - Placebo | −6.54 | 4.37 | [−17.06; 3.97] | 0.1351 |
| 80 ug/kg - Placebo | −12.23 | 4.55 | [−23.18; −1.28] | 0.0076 |
| 160 ug/kg - Placebo | −14.41 | 4.45 | [−25.11; −3.72] | 0.0013 |
| rFVIIa Combined - Placebo | −10.92 | 3.66 | [−19.73; −2.12] | 0.0030 |

Change in volumes are analysed by a generalised linear mixed model with subject and reader as random effects and volume at baseline, time from stroke to baseline CT, and time from baseline CT to dosing as fixed effects covariates

### Example 4

### Factor VIIa Administration to ICH Patients

Patients not developing oedema (oedema volume=0)−change in baseline to 72 hours

| Placebo | 40 μg/kg | 80 μg/kg | 160 μg/kg |
|---|---|---|---|
| 3 | 1 | 2 | 7 |
| 1.7% | 0.5% | 1.2% | 3.9% |

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A method for preventing or attenuating one or more complications of intracerebral haemorrhage (ICH), said method comprising administering to a patient in need thereof an effective amount for said preventing or attenuating of a first coagulation agent comprising Factor VIIa or a Factor VIIa equivalent.

2. A method as defined in claim 1, wherein said patient has experienced spontaneous or traumatic ICH.

3. A method as defined in claim 1, wherein said effective amount comprises at least about 40 μg/kg of said Factor VIIa or Factor VIIa equivalent.

4. A method as defined in claim 3, wherein said effective amount comprises at least about 80 μg/kg of said Factor VIIa or Factor VIIa equivalent.

5. A method as defined in claim 4, wherein said effective amount comprises at least about 120 μg/kg of said Factor VIIa or Factor VIIa equivalent.

6. A method as defined in claim 1, wherein said administering is carried out within about 24 hours of the occurrence of the ICH.

7. A method as defined in claim 6, wherein said administering is carried out within about 4 hours of the occurrence of the ICH.

8. A method as defined in claim 1, further comprising administering to the patient a second coagulation agent, wherein the amounts of said first and second coagulation agents together are effective in said preventing or attenuating.

9. A method as defined in claim 8, wherein the second coagulation agent is a coagulation factor.

10. A method as defined in claim 9, wherein said second coagulation agent is selected from the group consisting of: Factor VIII, Factor IX, Factor V, Factor XI, Factor XIII, a Factor VII or Factor VIIa different from the first coagulation agent, and combinations of any of the foregoing.

11. A method as defined in claim 8, wherein said second coagulation agent is an antifibrinolytic agent.

12. A method as defined in claim 11, wherein said antifibrinolytic agent is selected from the group consisting of PAI-1, aprotinin, ε-aminocaproic acid, tranexamic acid, and combinations of any of the foregoing.

13. A method as defined in claim 1, wherein said administering results in one or more of: a reduction in the number of days an ICH patient is hospitalized following ICH and a reduction in the risk of death in an ICH patient.

14. A method for preventing or attenuating one or more complications of ICH in a majority of ICH patients, which are carried out by: (i) administering to a group of ICH patients an amount effective for achieving the prevention or attenuation of Factor VIIa or a Factor VIIa equivalent; and (ii) observing a reduction in the frequency of occurrence of one or more complications of ICH among the group of patients who received Factor VIIa or a Factor VIIa equivalent relative to the frequency of occurrence of said complications that would have been expected in the same group of patients who had not received said Factor VIIa or Factor VIIa equivalent.
